(19) United States
(12) Patent Application Publication
(10) Pub. No.: US 2019/0125251 A1
(43) Pub. Date: May 2, 2019

(54) METHOD AND AN APPARATUS OF SENSING BRAIN SLEEP MODE

(71) Applicants: Xiaoping LI, Fukuoka-shi (JP); Qian XIA, Fukuoka-shi (JP); Jessica LI, Fukuoka-shi (JP); Joanna Le Xi LI, Fukuoka-shi (JP)

(72) Inventors: Xiaoping LI, Fukuoka-shi (JP); Qian XIA, Fukuoka-shi (JP); Jessica LI, Fukuoka-shi (JP); Joanna Le Xi LI, Fukuoka-shi (JP)

(21) Appl. No.: 16/173,503

(22) Filed: Oct. 29, 2018

Related U.S. Application Data
(60) Provisional application No. 62/578,463, filed on Oct. 29, 2017.

(51) Int. Cl. A61B 5/00

(52) U.S. Cl. CPC A61B 5/4815; A61B 5/4812; A61B 5/6824; A61B 5/7225; A61B 5/4809

(57) ABSTRACT

A method and apparatus manage a sleep mode of a brain by measuring a sleep depth as well as a sleep stage of the brain in the sleep mode. A damping/blocking level of a reticular formation is measured in descending motor cortex-emitted idle impulses to at least one part of a body.

### RELATED APPLICATIONS

This application claims the benefit of priority under 35 USC § 119(e) of U.S. Provisional Patent Application No. 62/578,463 filed 29 Oct. 2017, the contents of which is incorporated herein by reference in their entirety.

### TECHNICAL FIELD

For a brain sleep mode management system, the present application generally relates to a method and an apparatus of sensing the sleep depth and all sleep stages in the brain sleep mode by sensing the motor cortex emitted idle impulses that are descended from the brain to a part of the body.

### BACKGROUND

The brain consists of multiple functional sites providing various functions of the brain, of which the motor cortex functional site drives movements of the body parts by emitting neuronal electrical impulses via the nerve system to the body parts. The motor cortex is divided into multiple functional clusters. Each of the clusters drives a particular muscle movement in the body, and constantly emits electrical impulses with or without a movement onset signal from the brain to a muscle on a body part via the nerve system, in which the impulses without a movement onset signal are the “idle impulses” that result in the muscle tone for the body part posture, and the impulses with a movement onset signal result in the muscle contraction for the body part movement. The idle impulses can be detected from the nerves, muscles or skin on any part of the body when the brain is awake, by using electrodes with an amplifying circuit. For example, when the brain is awake, by placing electrodes with a signal amplifying system on the skin of the left wrist, electrical potential difference between the electrodes in contact with the skin varying against time can be detected when this part of the body is not carrying out any activities. The detected variation of the potential differences is a result of the idle impulses emitted from the right upper cluster of the motor cortex to the left wrist.

The brain has two working modes: the wake mode and the sleep mode. In the wake mode, the idle impulses emitted from all functional clusters of the motor cortex constantly reach all the body parts throughout the body via the nerve system, and the idle impulses can be measured from the nerves or skin on any part of the body. In the sleep mode, as sleep hormone is released in the brain, which inhabits neuronal activations. Under the influence of sleep hormone release, in brain reticular formation region, especially in the reticulospinal tracts of the descending reticular formation, the motor cortex emitted idle impulses are damped for descending to the body parts.

When the brain initially transforms from its wake mode to initial sleep mode—the so-called sleep onset stage, sleep hormone is lightly released in the reticular formation and the motor cortex emitted idle impulses are slightly damped for descending. This can be detected as a decrease in the amplitude of variation of the potential differences measured on a body part.

When the brain transforms further into its NRAM (non-rapid-eye-movement) sleep stage, sleep hormone is further released in the reticular formation and the motor cortex emitted idle impulses are further damped for descending. This can be detected as a further decrease in the amplitude of variation of the potential differences measured on a body part.

Before and during the brain transforms into its NRAM (non-rapid-eye-movement) sleep stage, during which the brain is in dreaming and sleep paralysis may occur, sleep hormone is such heavily released in the reticular formation that the motor cortex emitted idle impulses are fully blocked for descending. This can be detected as a disappearance of variation of the potential differences (a straight line rather than a wave form) measured on a body part.

By measuring the decrease of the amplitude of variation of the potential differences measured in the nerves, muscles or skin on a body part, the descending levels of the motor cortex emitted idle impulses from the reticular formation to all the body parts can be measured and the correlated brain sleep mode at all stages can be so sensed.

### SUMMARY

In the present invention, sleep depth as well as all sleep stages of the brain in its sleep mode is sensed by sensing under the influence of sleep hormone release the damping/blocking level of the reticular formation, especially the reticulospinal tracts of the descending reticular formation, in descending the motor cortex emitted idle impulses to all parts of the body.

In the present invention, one of the methods for measuring the sleep depth as well as all sleep stages of the brain in its sleep mode by measuring, under the influence of sleep hormone release, the damping/blocking level of the reticular formation, especially the reticulospinal tracts of the descending reticular formation, in descending the motor cortex emitted idle impulses to all parts of the body, is to measure the motor cortex emitted idle impulses that are descended from the brain reticular formation to a part of the body.

In the present invention, one of the methods for measuring the motor cortex emitted idle impulses that are descended from the brain reticular formation to a part of the body for measuring the sleep depth as well as all sleep stages of the brain in its sleep mode is to measure the amplitude of variation of the potential differences measured on a part of the body.

In the present invention, for example, one of the methods for measuring the motor cortex emitted idle impulses that are descended from the brain reticular formation to a part of the body for measuring the sleep depth as well as all sleep stages of the brain in its sleep mode is to measure the amplitude of variation of the potential differences measured on the left wrist of the body.

Particularly, there is provided a method of managing the brain sleep mode at the three stages: the sleep onset stage, the NREM sleep stage, and the REM sleep stage, by sensing the sleep depth in relation to, under the influence of sleep hormone release, the damping/blocking level of the reticular formation, especially the reticulospinal tracts of the descending reticular formation, in descending the motor cortex emitted idle impulses to all parts of the body, in which the sleep depth is defined by using a baseline measurement of the amplitude of variation of the potential differences measured on the left wrist of the body before the brain transforms from its wake mode to sleep mode, that is, the damping/blocking level of the reticular formation, especially the reticulospinal tracts of the descending reticular formation, in descending the motor cortex emitted idle impulses to all parts of the body is close to zero, wherein the sleep depth as well as all the sleep stages are defined and sensed using the following ratio:

Sleep Depth=the amplitude measured/the baseline value

where the smaller the value of Sleep Depth the deeper the level of sleep, and the order of the three stages of sleep mode in terms of the value of Sleep Depth is:

REM<NREM<Sleep Onset

That is, REM sleep is the deepest sleep.

In a further aspect of the present invention, there is provided an apparatus of managing the brain sleep mode by sensing the motor cortex emitted idle impulses that are descended from the brain reticular formation to a part of the body, comprising: 1) a signal acquisition unit having electrodes attached to the skin on a muscle of a body part for measuring the electrical potential differences between two locations on the skin, 2) a signal processing unit calculating the sensed sleep depth as well as brain sleep mode at all stages, 3) a data storage unit storing the processed results, 4) a controlling unit making decisions of and taking actions on switching on or switching off sleep modulation devices working in pair with the apparatus, 5) a transmitting unit transmitting data and controlling commands to external devices. The apparatus senses the motor cortex emitted idle potentials from the nerves in a muscle on a body part through its electrodes attached to the skin on the muscle, which receive the potential signals from the skin, as well as through its signal processing unit that collect idle potential signals from the electrodes, calculates the average amplitude of the idle potential over an interval of sleeping time as the sleep depth for the moment and records the value in its storage unit, and calculates the variation trend of sleep depth in terms of the variation slope, with the positive slope and negative slope indicating getting deep into sleep and getting less deep in sleep, respectively, and further, makes decisions on intervention of the sleep for getting deeper sleep, maintaining sleep depth, or waking up the brain from sleep, based on the calculated sleep depth variation slope, and furthermore, sends commands to control co-working devices for the sleeper in getting deeper sleep, maintaining sleep depth, or waking up.

For example, a wristband containing a signal acquisition unit with multiple electrodes, a signal processing unit, a data storage unit, a controlling unit and a wireless transmitting unit, in which the electrodes are attached to the skin of the left wristband receiving the idle impulses emitted from the right upper of the motor cortex in the brain, the received idle impulse signals are processed in the signal processing unit for sleep depth as well as the variation slopes of sleep depth throughout the duration of sleep, in which the sleep depth of a sleeper at a particular moment of time i during sleeping, SD(i), is calculated in the following steps:

$$SD ( i ) = \frac{Δ Vi}{Δ Vb}$$

where

$$Δ Vi = \frac{Δ Vi 1 + Δ Vi 2 + Δ Vi 3 + ⋯ + Δ Vin}{n}$$

is the average of the sampled potential differences at time i, and

$$Δ Vb = \frac{Δ Vb 1 + Δ Vb 2 + Δ Vb 3 + ⋯ + Δ Vbn}{n}$$

is the baseline potential differences and is the average of the sampled potential differences at the start of sleep when the brain is still in its wake mode.

The variation trend (the slope) of sleep depth at a particular moment of time i during a sleep, ηᵢ, is calculated in the following steps:

$${η}_{i} = {tan}^{- 1} \frac{[ SD ( i - 1 ) - SD ( i ) ]}{T ( i ) - T ( i - 1 )}$$

where SD (i−1) and SD (i) are the sleep depth value measured before the moment of Ti and the sleep depth value at the moment of Ti, respectively, and T(i−1) and T(i) are the time before the moment of Ti and the time at the moment of Ti, respectively.

In the present invention, the processed results of sleep depth, sleep stage, and variation trend of sleep depth will be saved in the data storage unit for been uploaded to external databases. Further, for sleep intervention towards wakefulness, when ηᵢ is negative, the controlling unit will trigger to turn on a co-working sleep modulation device for wakefulness, and then to turn it off when ηᵢ becomes positive for a period of time; for sleep induction towards deep sleep, when ηᵢ is changing from positive to zero and remains as zero for a period of time, the controlling unit will trigger to turn off a co-working sleep modulation device for deep sleep, and when ηᵢ is changing from zero to negative, the controlling unit will trigger to turn on the co-working sleep modulation device for deep sleep.

### BRIEF DESCRIPTION OF DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic diagram showing a method of managing the brain sleep mode by sensing the motor cortex emitted idle impulses that are descended by the reticular formation to the body parts, with an apparatus equipped with electrodes attached to the left wrist measuring the variations of electrical potential differences resulted from the right upper motor cortex cluster emitted idle impulses that are descended by the reticular formation to the left wrist.

FIG. 2 is a schematic diagram showing the function blocks of an apparatus of managing the brain sleep mode by sensing the motor cortex emitted idle impulses that are descended by the reticular formation to the body parts, having an electrical potential signal acquisition unit, a signal processing unit, a data storage unit, a controlling unit and a transmitting unit.

### DETAILED DESCRIPTION OF THE INVENTION

Some embodiments of the present invention will be described hereinafter with reference to the accompanying drawings.

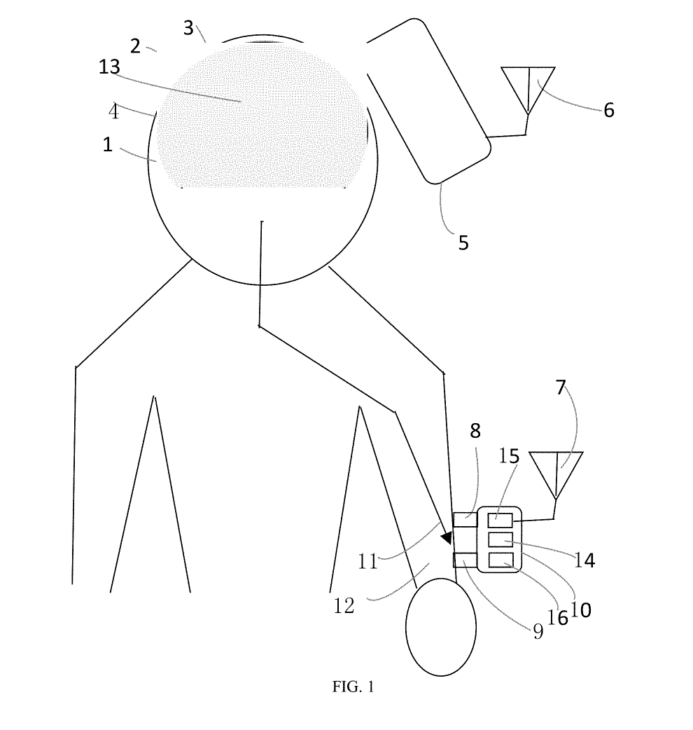

In an embodiment, as shown in FIG. 1, a method of sensing the sleep depth and all sleep stages of the brain sleep mode in a brain 1, is to sense the damping/blocking level of the reticular formation 4 in brain 1, under the influence of sleep hormone release in brain 1, in descending the right upper cluster 3 of motor cortex 2 emitted idle impulses 13, by sensing the right upper cluster 3 of motor cortex 2 emitted idle impulses 11 that are descended by the reticular formation 4 to the left wrist 12, with a sleep mode management apparatus 10 having electrodes 8 and 9 attached to the left wrist 12 to collect electrical potential signals resulted from the right upper cluster 3 of motor cortex 2 emitted idle impulses 11 and to process the collected data with its signal processing unit 16 and to pass the processed results to data storage unit 14 and controlling unit 15 for transmitting the results via transmitting unit 7 to external devices, including co-working sleep modulation device 5 that receives commands from controlling unit 15 via receiving unit 6.

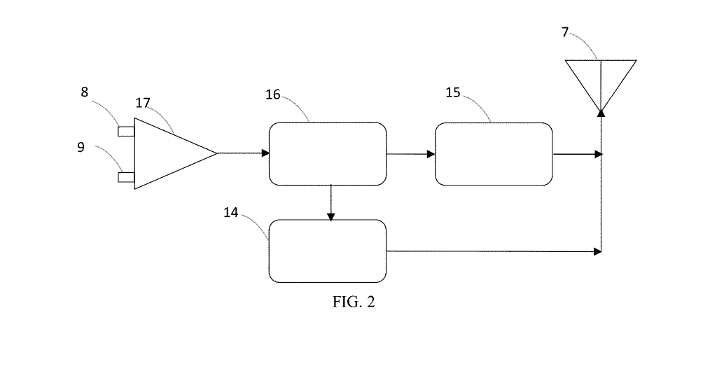

In another embodiment, as shown in FIG. 2, the function blocks of an apparatus of managing the brain sleep mode by sensing the motor cortex emitted idle impulses that are descended by the reticular formation to the body parts, having an electrical potential signal acquisition unit 17, a signal processing unit 16, a data storage unit 14, a controlling unit 15 and a transmitting unit 7. The signal acquisition unit 17 has electrodes 8 and 9 attached to the skin of a part of the body to collect electrical potential signals resulted from the reticular formation descended motor cortex emitted idle impulses, to determine the potential differences, and to pass the results to signal processing unit 16. The signal processing unit 16 processes the potential differences in variation against time for the results of sleep depth, sleep depth slope (variation trend of the sleep depth), and passes the results to data storage unit 14 and controlling unit 15 for transmitting to external databases and for controlling co-working sleep modulation devices, via transmitting unit 7.

1.-12. (canceled)

13. A method of managing a sleep mode of a brain, comprising:
determining a sleep depth as well as a sleep stage of the brain in the sleep mode by measuring a release level of sleep hormone in the brain.

14. The method of claim 13, wherein the measuring a release level of sleep hormone in the brain comprises measuring a damping/blocking level of the brain reticular formation in descending motor cortex emitted idle impulses to at least one part of a body, in which the damping/blocking level in a wake mode is close to zero.

15. The method of claim 14, wherein the brain reticular formation is reticulospinal tracts of the brain reticular formation.

16. The method of claim 14, wherein the measuring the damping/blocking level comprises measuring the motor cortex emitted idle impulses that are descended from the brain reticular formation to the at least one part of the body.

17. The method of claim 16, wherein the measuring the motor cortex emitted idle impulses comprises measuring an amplitude of variation of at least one potential difference measured on the at least one part of the body.

18. The method of claim 14, wherein the at least one part of the body is left wrist of the body.

19. The method of claim 13, wherein the sleep stage is one of stages comprising a sleep onset stage, a NREM sleep stage, and a REM sleep stage.

20. The method of claim 19, wherein the sleep stage is measured by measuring the sleep depth.

21. The method of claim 17, wherein the sleep depth is defined by using a baseline measurement of the amplitude of variation of the potential difference measured on one part of the body before the brain transforms from a wake mode to the sleep mode.

22. The method of claim 21, wherein the sleep depth follows: the sleep depth=the amplitude measured/the baseline measurement, where the smaller a value of the sleep depth the deeper a level of sleep.

23. The method of claim 19, wherein an order of the stages in terms of values of sleep depths follows: the sleep onset stage>the NREM sleep stage>the REM sleep stage, where the REM sleep stage corresponds to the deepest sleep.

24. The method of claim 13, further comprising determining a variation trend of the sleep depth; and controlling a sleep modulation device based upon the determined variation trend of the sleep depth.

25. An apparatus for managing a sleep mode of a brain, comprising:
a signal acquisition unit configured to measure a release level of sleep hormone in the brain;
a signal processing unit configured to determine, according to the release level of sleep hormone, a sleep depth as well as a sleep stage of the brain in the sleep mode, and further to determine a variation trend of the sleep depth;
a data storage unit configured to receive and to record the results of the sleep depths, sleep stages, variation trends of the sleep depth, and to send out the results to external databases;
a controlling unit configured to receive the results from the signal processing unit and to control co-working sleep modulation devices;
a transmitting unit configured to transmit data and controlling commands from the data storage unit and controlling unit to external databases and co-working sleep modulation devices.

26. The apparatus of claim 25, wherein the signal acquisition unit is configured to measure the release level of sleep hormone by measuring a damping/blocking level of the brain reticular formation in descending motor cortex emitted idle impulses to at least one part of a body.

27. The apparatus of claim 26, wherein the signal acquisition unit is configured to measure the damping/blocking level by measuring the motor cortex emitted idle impulses that are descended from the brain reticular formation to the at least one part of the body.

28. The apparatus of claim 27, wherein the signal acquisition unit comprises a potential sensing circuit having electrodes attached to a skin on a muscle of at least one body part, the potential sensing circuit being configured to measure an amplitude of variation of at least one potential difference measured on the at least one part of the body, the signal acquisition unit being configured to measure the motor cortex emitted idle impulses according to the measured amplitude of variation.

29. The apparatus of claim 28, wherein the signal processing unit is configured to determine the sleep depth by using a baseline measurement of the amplitude of variation of the potential difference measured on one part of the body before the brain transforms from a wake mode to the sleep mode.

30. The apparatus of claim 29, wherein the sleep depth follows: the sleep depth=the amplitude measured/the baseline measurement, where the smaller a value of the sleep depth the deeper a level of sleep.

31. The apparatus of claim 25, wherein the signal processing unit is configured to further determine a variation trend of the sleep depth, and the apparatus further comprises a controlling unit configured to control, via the transmitting unit, a sleep modulation device working in pair with the apparatus based upon the determined variation trend of the sleep depth.

32. The apparatus of claim 25, wherein the data storage unit is configured to receive and to record the signal processing unit processed results of the sleep depths, sleep stages, and variation trends of the sleep depth, and to send the results to external databases via the transmitting unit.
